SEQUENCE LISTING (9 sequences)

SEQ ID NO: 1 - DNA, 19 bp, Transposon Tn5
ctgactcttatacacaagt

SEQ ID NO: 2 - DNA, 19 bp, Transposon Tn5
ctgtctcttgatcagatct

SEQ ID NO: 3 - DNA, 19 bp, Transposon Tn5
ctgtctcttatacacatct

SEQ ID NO: 4 - DNA, 19 bp, Transposon Tn5
agatgtgtataagagacag

SEQ ID NO: 5 - DNA, 137 bp, Artificial
  cloning plasmid pCI and transposon Tn5 sequence
cggacaggtatccggtaagcggcagggtcggaacaggagctgtctcttatacacatctagggtgtggaaagttttggtcatgagaattcagatgtgtataagagacaggaacaggagagcgcacgagggagcttcca

SEQ ID NO: 6 - DNA, 137 bp, Artificial
  cloning vector pCI and transposon Tn5 sequence
accggataaggcgcagcggtcgggctgaacgggggctgtctcttatacacatctgaattctcatgaccaaaagactttccacaccctagatgtgtataagagacaggaacggggggttcgtgcacacagcccagctt

SEQ ID NO: 7 - DNA, 136 bp, Artificial
  cloning vector pCI and transposon Tn5 sequence
gcggtatttcacaccgcatatggtgcactcctgtctcttatacacatctagggtgtggaaagtccccaggcttggtcatgagaattcagatgtgtataagagacagggtgcactctcagtacaatctgctctgatg

SEQ ID NO: 8 - DNA, 1608 bp, Transposon Tn5
atgataacttctgctcttcatcgtgcggccgactgggctaaatctgtgttctcttcggcggcgctgggtgatcctcgccgtactgcccgcttggttaacgtcgccgcccaattggcaaaatattctggtaaatcaataaccatctcatcagagggtagtaaagccgcccaggaaggcgcttaccgatttatccgcaatcccaacgtttctgccgaggcgatcagaaaggctggcgccatgcaaacagtcaagttggctcaggagtttcccgaactgctggccattgaggacaccacctctttgagttatcgccaccaggtcgccgaagagcttggcaagctgggctctattcaggataaatcccgcggatggtgggttcactccgttctcttgctcgaggccaccacattccgcaccgtaggattactgcatcaggagtggtggatgcgcccggatgaccctgccgatgcggatgaaaaggagagtggcaaatggctggcagcggccgcaactagccggttacgcatgggcagcatgatgagcaacgtgattgcggtctgtgaccgcgaagccgatattcatgcttatctgcaggacaaactggcgcataacgagcgcttcgtggtgcgctccaagcacccacgcaaggacgtagagtctgggttgtatctgtacgaccatctgaagaaccaaccggagttgggtggctatcagatcagcattccgcaaaagggcgtggtggataaacgcggtaaacgtaaaaatcgaccagcccgcaaggcgagcttgagcctgcgcagtgggcgcatcacgctaaaacaggggaatatcacgctcaacgcggtgctggccgaggagattaacccgcccaagggtgagaccccgttgaaatggttgttgctgaccagcgaaccggtcgagtcgctagcccaagccttgcgcgtcatcgacatttatacccatcgctggcggatcgaggagttccataaggcatggaaaaccggagcaggagccgagaggcaacgcatggaggagccggataatctggagcggatggtctcgatcctctcgtttgttgcggtcaggctgttacagctcagagaaagcttcacgccgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctgggaaggttgggaagccctgcaaagtaaactggatggctttcttgccgccaaggatctgatggcgcaggggatcaagatcgtcgactccaccaacgagaatgctaatacaccagctgcccgtcttcacagattcaagaacaagggaaaagacagtacagaaatgaggcgtcgcagaatagaggtcaatgtggagctgaggaaagctaagaaggatgaccagatgctgaagaggagaaatgtaagctcatttcctgattga

SEQ ID NO: 9 - DNA, 1554 bp, Transposon Tn5
atgataacttctgctcttcatcgtgcggccgactgggctaaatctgtgttctcttcggcggcgctgggtgatcctcgccgtactgcccgcttggttaacgtcgccgcccaattggcaaaatattctggtaaatcaataaccatctcatcagagggtagtaaagccgcccaggaaggcgcttaccgatttatccgcaatcccaacgtttctgccgaggcgatcagaaaggctggcgccatgcaaacagtcaagttggctcaggagtttcccgaactgctggccattgaggacaccacctctttgagttatcgccaccaggtcgccgaagagcttggcaagctgggctctattcaggataaatcccgcggatggtgggttcactccgttctcttgctcgaggccaccacattccgcaccgtaggattactgcatcaggagtggtggatgcgcccggatgaccctgccgatgcggatgaaaaggagagtggcaaatggctggcagcggccgcaactagccggttacgcatgggcagcatgatgagcaacgtgattgcggtctgtgaccgcgaagccgatattcatgcttatctgcaggacaaactggcgcataacgagcgcttcgtggtgcgctccaagcacccacgcaaggacgtagagtctgggttgtatctgtacgaccatctgaagaaccaaccggagttgggtggctatcagatcagcattccgcaaaagggcgtggtggataaacgcggtaaacgtaaaaatcgaccagcccgcaaggcgagcttgagcctgcgcagtgggcgcatcacgctaaaacaggggaatatcacgctcaacgcggtgctggccgaggagattaacccgcccaagggtgagaccccgttgaaatggttgttgctgaccagcgaaccggtcgagtcgctagcccaagccttgcgcgtcatcgacatttatacccatcgctggcggatcgaggagttccataaggcatggaaaaccggagcaggagccgagaggcaacgcatggaggagccggataatctggagcggatggtctcgatcctctcgtttgttgcggtcaggctgttacagctcagagaaagcttcacgccgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctgggaaggttgggaagccctgcaaagtaaactggatggctttcttgccgccaaggatctgatggcgcaggggatcaagatcgtcgactcagaagaaatgccatctagtgatgatgaggctactgctgactctcaacattctactcctccaaaaaagaagagaaaggtagaagaccccaaggactttccttcagaattgctaagttga

(19) United States
(12) Patent Application Publication
(10) Pub. No.: US 2003/0143740 A1
(43) Pub. Date: Jul. 31, 2003

(54) Processes for transposase mediated integration into mammalian cells

(76) Inventors: Christine Wooddell, Madison, WI (US); Hans Herweijer, Madison, WI (US); Jon A. Wolff, Madison, WI (US)

Correspondence Address: Mark K. Johnson, Mirus, 505 South Rosa Road, Madison, WI, 53719, US

(21) Appl. No.: 10/272,552

(22) Filed: Oct. 15, 2002

Related U.S. Application Data
(60) Provisional application No. 60/329,474, filed on Oct. 15, 2001.
(60) Provisional application No. 60/344,865, filed on Nov. 8, 2001.

(51) Int. Cl. C12N 15/85

(52) U.S. Cl. 435/455; 435/473

(57) ABSTRACT

We disclose compositions and processes for transferring a nucleic acid into a mammalian cell utilizing a transposase to achieve nonviral integration of exogenous nucleic acid into the chromosomal DNA of the cell.

This application claims priority benefit of U.S. Provisional Applications Serial No. 60/329474 filed Oct. 15, 2001 and Serial No. 60/344,865 filed Nov. 8, 2001.

### FIELD OF THE INVENTION

The present invention relates to compositions and processes for delivery of a transposon integration complex to a mammalian cell and integration of a nucleic acid into the genome of the cell.

### BACKGROUND

Microbial transposition systems are well established tools for genetics and genome analysis. Transposition into eukaryotic cells occurs naturally through viral infection and via Tc1/mariner type elements. Integration capabilities of retroviral vectors and adeno-associated viral vectors have been studied as candidates for gene transfection.

The ability of current retroviral or adeno-associated viral vectors to integrate into mammalian genomes increases their utility for enabling prolonged expression in dividing cells both ex vivo and in vivo. However, these vectors have limited insert capacity: retroviral and adeno-associated viral vectors can respectively carry only up to ten and five kilobases of foreign DNA. This limitation not only restricts the size of a cDNA that can be expressedbut also seriously restricts the amount of regulatory sequence that can be delivered with the cDNA. The ability to use large genes with more complete transcriptional and translational cis regulatory sequences would aid the development of gene therapy. The fuller complement of regulatory sequences would also enable the expression of foreign genes to be under better physiological, tissue-specific, and developmental control. For example, a 12-kb fragment of the 5′- flanking region of the albumin gene was shown to enable higher levels of liver expression than a 0.3-kb fragment [Pinkert et al. 1987]. Viral cis sequences can also adversely affect foreign gene expression.

DNA transposition is an important mechanism in the rearrangement of genomes and horizontal gene transfer in prokaryotic as well as eukaryotic cells. The dissemination of antibiotic resistance genes in bacteria is largely due to transposons. A transposable element has short inverted repeats flanking an intervening DNA sequence. A transposase or integrase binds to these elements, excises the transposon from one location in the DNA, and inserts it (including the inverted repeats with the intervening sequence) into another location. A characteristic of integration by transposable elements in both prokaryotes and eukaryotes is the duplication of a short segment of genomic sequence flanking the insertion sites. The duplications are characteristic for each transposon: they are 9 bp for Tn5, 4 bp for murine leukemia virus, and 2 bp for the Tc1/mariner family duplications. Retroviruses such as the human immunodeficiency virus also integrate into the human genome.

The frequency of transposition is very low for most transposons, which use complex mechanisms to limit activity. Tn5 transposase, for example, utilizes a DNA binding sequence that is suboptimal and the C-terminus of the transposase interferes with DNA binding. Mechanisms involved in Tn5 tranposition have been carefully characterized by Reznikoff and colleagues. Tn5 transposes by a cut-and-paste mechanism. The transpos on has two pair of 19 bp elements that are utilized by the transposase: outside elements (OE) and inside elements (IE). One transposase monomer binds to each of the two elements that are utilized. After a monomer is bound to each end of the transposon, the two monomers dimerize, forming a synapse. Vectors with donor backbones of at least 200 bp, but less than 1000 bp, are most functional for transposition in bacteria. Transposon cleavage occurs by trans catalysis and only when monomers bound to each DNA end are in a synaptic complex. Tn5 transposes with a relaxed target site selection and can therefore insert into target DNA with little to no target sequence specificity.

The natural downregulation of Tn5 transposition was overcome by selection of hyperactive transposase and by optimizing the transposase-binding elements [Yorket al. 1998]. A mosaic element (ME), made by modification of three bases of the wild type OE, led to a 50-fold increase in transposition events in bacteria as well as cell-free systems. The combined effect of the optimized ME and hyperactive mutant transposase is estimated to result in a 100-fold increase in transposition activity. Goryshin et al showed that preformed Tn5 transposition complexes could be functionally introduced into bacterial or yeast by electroporation [Goryshin et al. 2000]. Linearization of the DNA, to have inverted repeats precisely positioned at both ends of the transposon, allowed Goryshin and coworkers to bypass the cutting step of transposition thus enhancing transposition efficiency.

Cell-free systems for intermolecular transposition have been developed from Tn5 [Goryshin and Reznikoff 1998], Tn7 [Gwinn et al. 1997], Mu [Haapa et al. 1999], and the yeast Ty1 virus-like particles [Devine and Boeke 1994]. Sleeping Beauty transposase,which has been shown to work in mammalian cells, requires inverted repeat elements of ˜230 base pairs at each side of the DNA to transpose and must be expressed in the mammalian cell.

### BRIEF DESCRIPTION OF FIGURES

FIG. 1. The transposon components of plasmids pNeo-Tn (pMIR3), pEGFP-Tn (pMIR151), pSEAP-Tn (pMIR136), and pNeo/siRNA-Tn (pMIR246) including the 19 bp mosaic elements (ME, black boxes) are shown The ME's are inverted repeats. The transposons of each of these plasmids include the SV40 promoter driving the neomycin resistance gene and a prokaryotic promoter to allow for kanamycin resistance in bacterial cells. In plasmids pNeo-Tn, pEGFP-Tn, and pSEAP-Tn the bacterial origin of replication is included in the transposon. In pNeo/siRNA-Tn, the bacterial origin is outside of the transposon elements. Blunt-ended transposons were released from each of the plasmids by digestion with restriction enzyme PshA I. Just internal to each of the ME's is the restriction site indicated. Prokaryotic promoter (P_{Kan}), eukaryotic promoters (P_{CMV}, P_{SV40} and P_{UbC}), SV40 or HSV TK polyA sequences (pA), the bacterial origin of replication (ori) and the fl origin or replication (fl ori) are shown.

FIG. 2. Formation of Tn5 integrator complexes. Lane 1: MassRuler DNA Ladder Mix molecular weight markers (MBI Fermentas). Lane 2: Neo-Tn transposon+vector backbone fragment. pNeo-Tn is 2,913 bp. Lane 3: supercoiled target plasmid, pUC18. Lane 4: Tn5 transposase/Neo-Tn synaptic complexes. Lane 5: SDS dissociated synaptic complexes. Lane 6: integration products into SEAP-Tn that form when magnesium is added to the synaptic complexes. Lane 7: integration products that result from addition of pUC18 to preformed synaptic complexes in the presence of magnesium. (TN DNA=Neo-Tn, Tnp=Tn5 transposase)

FIG. 3. Synaptic complexes formed with SEAP-Tn and EGFP-Tn is dependent on the presence of mosaic elements. Lane 1: molecular weight markers. Lane 2: SEAP-TN with mosaic elements removed. Lane 3: SEAP-Tn/Tn5 transposase integrator complexes. Lane 4: SEAP-Tn with mosaic elements removed+Tn5 transposase, no integrator complexes formed. Lane 5: EGFP-Tn alone. Lane 6 and 7: EGFP-Tn/Tn5 transposase integrator complexes. Lane 8: EFGP-Tn with mosaic elements removed +Tn5 trarsposase, no integrator complexes formed. (TN DNA=SEAP-Tn or EGFP-Tn, ME=mosaic element, Tnp=Tn5 transposase)

FIG. 4. Tn5 transposase is active in the presence of mammalian cell transfection reagents. Lane 1: molecular weight markers. Lane 2: SEAP-Tn. Lane 3: supercoiled pUC18 target DNA. Lane 4 SEAP-Tn+Tn5 transposase. Lane 5: integration into SEAP-Tn Lane 6: SEAP-Tn integration into pUC18. Lane 7: SEAP-Tn integration into pUC18 in presence of the transfection reagent Trans-IT LT1. Lane 8: SEAP-Tn integration into pUC18 in presence of the transfection reagent Trans-IT Insecta. Lane 9: SEAP-Tn integration into pUC18 in presence of the transfection reagent poly(ethyleneimine). (TN DNA=SEAP-Tn, Tnp=Tn5 transposase, LT TransIT LT1 , In=TransIT Insecta, PE=poly(ethyleneimine))

FIG. 5. Delivery of integrator complexes to 3T3 cells with TransIT LT1 transfection reagent. NIH-3T3 cells were transfected with: 2 μg supercoiled pNeo-Tn (uncut-2); 2, 5 or 10 μg PshA I linearized pNeo-Tn (linear-2, linear-5, linear-10); or 1, 2.5 or 5 μg PshA I linearized pNeo-Tn in preformed complexes with Tn5 transposase (TN-1, TN-2.5 or TN-5). Graphed are the number of G418-resistant colonies per plate of 500-fold dilution from transfected cells.

### SUMMARY

In a preferred embodiment, we describe a process for non-viral integration of a nucleic acid into the genome of a mammalian cell comprising: making a transposon consisting of a nucleic sequence flanked on either side by a Tn5 element, forming a Tn5 integrator complex between the transposon and a Tn5 transposase, and delivering the complex to a mammalian cell wherein the transposon is integrated into chromosomal DNA. Any nucleic sequence that is flanked by Tn5 elements may be integrated into a mammalian cell chromosome. The nucleic acid sequence may include a therapeutic gene or a marker gene or other expression cassette or marker sequence. The nucleic acid sequence may also include sequences that affect expression of the gene. A preferred transposase is a hyperactive Tn5 transposase. Integration of the sequence into the genome of the cell may provide long term persistence of the sequence in the cell. Integration may also provide long term expression of a therapeutic gene.

In a preferred embodiment, any nucleic acid sequence that is flanked on either side by inverted repeat sequences to which Tn5 transposase can bind maybe used in the process. A preferred flanking sequence is the 19 base pair Mosaic element (ME). Other preferred flanking sequences are the outside Tn5 element (outside ends) and the inside Tn5 element (inside ends). The nucleic acid sequence plus the flanking sequence together are called the transposon. The transposon may by linear or circular. The transposon may be flanked by additional sequences such as in a plasmid. The plasmid may be linear, circular or supercoiled.

In a preferred embodiment, a Tn5 integrator complex is formed in a container outside the cell and delivered to a mammalian cell. The Tn5 integrator complex is formed by complexing the Tn5 transposase with a transposon in conditions that allow complex formation. The conditions may inhibit transposition, such as in buffer lacking magnesium, until the complex is delivered to the cell. The Tn5 integrator complex may be formed on a transposon that is linear or circular. The transposon may comprise all or a portion of the nucleic acid in the integrator complex. A preferred Tn5 transposase is a hyperactive transposase. A preferred hyperactive transposase is the EK54/MA56/LP372 mutant Tn5 transposase. In a preferred embodiment, the transposase may be modified to contain a nuclear localization signal. The use of preformed Tn5 integrator complexes bypasses the need to express an integrase in the target host, and thereby increases stability of the transposed element.

In a preferred embodiment, compositions comprising transposase integrator complexes and mammalian cell transfection reagents, and processes using such compositions to deliver a transposon integrator complex to a mammalian cell in vivo or in vitro for the purposes of integrating a nucleic acid sequence into a chromosome of the cell are described.

In a preferred embodiment, the present invention provides a process for delivering a transposase/transposon integrator complex to an animal cell comprising; forming an integrator complex, preparing a composition comprising mixing a transfection reagent with the integrator complex in a solution, associating the composition with a mammalian cell, and delivering the integrator complex to the interior of the cell. The transposon is then integrated into the genome of the cell. Preferred transfection reagents include TransIT LT1, TransIT Insecta, poly(ethyleneimine). Other transfections reagents that may be used include cationic polymers such as and polylysine, cationic polymer conjugates, cationic proteins, liposomes, cationic lipids and combinations of these.

In another preferred embodiment, a Tn5 integrator complex may be delivered to a mammalian cell by co-transfecting the cell with a nucleic acid containing a transposon and a nucleic acid containing an expressible Tn5 transposase gene wherein the transposase is expressed and forms an integrator complex on the transposon, and the transposon is integrated into a chromosome. The transposon and the transposase gene may be on the same or different nucleic acid molecules. The nucleic acid containing the transposon may be circular or linear. The nucleic acid containing the Transposase gene contains the coding region downstream of a promoter in a mammalian expression cassette that is active in the target cell. It is preferable to use a promoter that is rapidly down regulated to limit expression of the transposase. A preferred Tn5 transposase gene is a gene encoding a hyperactive Tn5 transposase. A preferred hyperactive Tn5 transposase is the EK54/MA56/LP372 hyperactive Tn5 transposase. In a preferred embodiment, the transposase gene may be modified to encode a transposase with a nuclear localization signal. Any method in the art of transferring nucleic acid to a mammalian cell may be used to deliver the nucleic acid to the cell. These methods include viral vectors comprising: adenovirus, adeno-associated virus (AAV), retrovirus and lentivirus vectors [Blomer et al. 1997]; non-viral methods comprising: cationic polymers such as PEI and polylysine, cationic polymer conjugates, cationic proteins, liposomes, cationic lipids and combinations of these; and other means including the biolistic “gun”, electroporation, microinjection, and naked DNA. The cell may be in vivo, in situ, ex vivo, or in vitro.

In a preferred embodiment, the process can be used to integrate a therapeutic gene into the genome of a mammalian cell. Therapeutic genes include genes that encode a therapeutic RNA or protein or genes that effect expression of endogeous genes. Examples of genes that affect endogenous genes include: siRNA, antisense, and ribozymes.

In a preferred embodiment, the cell can be a primary or secondary cell which means that the cell has been maintained in culture for a relatively short time after being obtained from an animal. These include, but are not limited to, primary liver cells and primary muscle cells and the like. The process may be used to integrate a therapeutic gene into a chromosome of a mammalian cell that is ex vivo to produce genetically modified cells such as embryonic stem cells, bone marrow stem cells, pluripotent precursor blood cells, precursor neuronal cells, lymphocytes, fibroblasts, keratinocytes, and myoblasts. The genetically-modified cell carrying the integrated nucleic acid may then be re-implanted or transplanted into a mammal.

In a preferred embodiment, the cell can be a mammalian cell that is maintained in tissue culture such as cell lines that are immortalized or transformed. These include a number of cell lines that can be obtained from American Type Culture Collection (Bethesda) such as, but not limited to: 3T3 (mouse fibroblast) cells, Rat1 (rat fibroblast) cells, CHO (Chinese hamster ovary) cells, CV-1 (monkey kidney) cells, COS (monkey kidney) cells, 293 (human embryonic kidney) cells, HeLa (human cervical carcinoma) cells, HepG2 (human hepatocytes) cells, and the like.

In another preferred embodiment, the cell can be a mammalian cell that is within the tissue in situ or in vivo meaning that the cell has not been removed from the tissue or the animal.

In a preferred embodiment, the process may be used to provide random insertional mutagenesis, wherein integration of an exogenous nucleic acid into a chromosome disrupts an endogenous gene or inserts a molecular tag into a chromosome. Integration into a gene coding region can disrupt gene function and facilitate study of the gene. Integration of molecular tags can facilitate cloning, sequencing, or identification by providing a marker in a chromosome.

In a preferred embodiment, the process may be used to identify enhancer elements in the genome of a mammalian cell (enhancer-trapping) wherein; a transposon is created with a weak promoter and a reporter gene, a Tn5 integrator complex containing the transposon is delivered to a cell, and the transposon is integrated into the genome of the cell. Activity of the reporter gene is then monitored is response to different experimental conditions. A reporter gene in a transposon that is integrated near an enhancer will be expressed in conditions where the enhancer is active. Insulator sequences can be included to further define the location of the enhancer relative to the transposon insertion point. An insulator sequence may be placed on either side of the reporter gene in the transposon.

### DETAILED DESCRIPTION

The bacterial Tn5 transposase has been effectively used to generate transposition into the genome of bacteria and yeast. Surprisingly, we have found that Tn5 transposase is also active in mammalian cells. We now show that an integration system based upon the Tn5 transposase enables integration of exogenous nucleic acid sequences into the genome of mammalian cells. The system requires the delivery of an integrator complex to a cell. The integrator complex comprises a transposon and Tn5 transposase in a synaptic complex. A synaptic complex is formed when transposase monomers bind to each of two specific end-binding sequences on the transposon and then associate to bring the proteins and the two ends of the transposon together.

The Tn5 integration system requires two components: Tn5 transposase protein and a suitable transposon. Other components, such a transfection reagents or other delivery reagents or methods may also be used. The Tn5 transposase may be purified from natural sources or it may be recombinant protein produced in vitro or it may be synthesized by methods known in the art. Recombinant Tn5 transposase may be expressed in bacterial, yeast, insect or mammalian cells. The transposase may also be produced in cell-free expression systems. The transposase may also be expressed in a mammalian cell that is the target for the intended transposon integration event. The Tn5 transposase may have a wild-type amino acid sequence or it may have a modified amino acid sequence. Modifications include mutations that affect the activity or stability of the transposase or add functionality to the transposase. Specifically, mutations that enhance activity of the Tn5 transposase to produce a hyperactive protein are useful for the invention. Such mutations include the glutamate₅₄-to-lysine (EK54), methionine₅₆ to alanine (MA56), and leucine₃₇₂ to proline (LP372) mutations and combinations of these mutations (EK54,MA56,LP372 Tn5 transposase). Modifications that add functionality to the transposase include cell targeting or nuclear localization signals. The presence of a nuclear localization signal may facilitate entry of the transposase into the mammalian cell nucleus and enhance activity in both dividing and non-dividing cells.

The Tn5 transposon comprises any nucleic acid sequence that is flanked on both sides by inverted repeat sequences to which Tn5 transposase can bind and form a synaptic complex. These sequences are called the end-binding sequences or Tn5 elements and define the boundary of the transposon. Tn5 elements are typically ˜19 base pair sequences. Known elements include: outside elements, 5′-CTGACTCTTATACACAAGT-3′ (SEQ ID 1); inside elements, 5′-CTGTCTCTTGATCAGATCT-3′ (SEQ ID 2); and the mosaic element, 5′-CTGTCTCTTATACACATCT-3′ (SEQ ID 3). The transposon is thus defined as the nucleic acid sequence containing the Tn5 elements together with all of the nucleic acid sequence between the elements. The transposon may exist as a linear nucleic acid molecule with the Tn5 elements at the termini. Alternatively, the transposon may exist within a larger nucleic acid molecule such as a plasmid. Sequence outside the Tn5 elements is separated from the transposon during the transposition process. The transposon, including the Tn5 elements, is integrated into the target nucleic acid by the transposase.

The transposon may contain any nucleic acid sequence. The invention may be used to integrate therapeutic genes, siRNA genes, genes containing RNA polymerase III promoters (including the U6 promoter), reporter genes, marker or tag sequences, etc. More than one gene can be present on the transposon. For siRNA expression cassettes, the siRNA strands can either be transcribed as sense and anti-sense strands from separate promoters [Miyagishi and Taira 2002] or from a single promoter as a hairpin RNA that contains both sense and anti-sense [Sui et al. 2002]. The transposon may be used to integrate large DNA molecules, up to 10 kb or larger, into the genome of a mammalian cell.

The utility of the Tn5 integration system to integrate exogenous nucleic acid into the genome of a mammalian cell requires that the Tn5 integrator complex be delivered to the cell. The integration complex can be delivered to the cell as a preformed complex. Alternatively the integrator complex can be formed in the cell from transposase that is expressed in the cell, such as from a delivered expressible gene, and a delivered transposon.

The preformed complex consists of the transposon in a synaptic complex with a transposase dimer. Preformed integrator complexes can be made from purified transposon and transposase in a wide variety of buffers provided the buffer allows the formation of synaptic complexes. Buffers without divalent cations, particularly magnesium, may provide more stable formation of synaptic complexes prior to cell delivery. The integration reaction, but not the formation of a synaptic complex, requires the presence of magnesium[Goryshin et al. 2000]. Thus, in the absence of divalent cations, more stable synaptic complexes can be formed in a tube prior to delivery to a mammalian cell.

A number of transfection reagents have been developed for delivery of DNA to cells. These reagents have generally not been shown to be effective for delivery of proteins to cells. We have shown however, that several transfection reagents are effective in delivery of Tn5 transposase protein-nucleic acid complexes to mammalian cells. We have thus shown that the stability of the protein-DNA interactions and transposition competence of the complexes are maintained when associated with cationic transfection reagents The transfection reagent is associated with the integrator complex in an appropriate buffer and then associated with the target cell. The complex is then delivered to the cell and the transposon in integrated into the genome of the cell. Transfection reagents may also be useful in the delivery of other nucleic acid-protein complexes.

As an alternative to delivering preformed transposase-DNA complexes to cells, the transposase may be expressed separately from an expression cassette co-transfected with the transposon. The expression cassette may be a DNA expression cassette, such as a plasmid or an RNA expression cassette, such as an mRNA. The cassette contains the coding sequence for the transposase along the with regulatory sequences appropriate for expression in the target cell. We have shown that when such an expression cassette is co-transfected, along with a transposon, into a mammalian cell, the transposase is expressed, forms am integrator complex with the transposon, and integrates the transposon into DNA in the cell. The transposon may be integrated into genomic (chromosomal) DNA or extra-chromosomal DNA in the cell. It may be beneficial for the Tn5 transposase to be expressed in the mammalian cell from a promoter that is rapidly shut down. Thus, the transposase is expressed and mediates integration, but there is not continued expression of the transposase, thereby limited transposition after the initial integration event.

A hyperactive transposase is a transposase that has increased activity relative to the wild-type, or naturally occurring transposase.

The term nucleic acid, or polynucleotide, is a term of art that refers to a polymer containing at least two nucleotides. Natural nucleotides contain a deoxyribose (DNA) or ribose (RNA) group, a phosphate group, and a base. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasse s any of the known base analogs of DNA and RNA. Nucleotides are the monomeric units of nucleic acid polymers and are linked together through the phosphate groups. Polynucleotides with less than 120 monomeric units are often called oligonucleotides. The term polynucleotide includes deoxyribonucleic acid (DNA) and ribonucleic acid (RNA). Natural polynucleotides have a ribose-phosphate backbone. An artificial or synthetic polynucleotide is any polynucleotide that is chemically polymerized and contains the same or similar bases but may contain a backbone of a type other than the natural ribose-phosphate backbone. These backbones include, but are not limited to: PNAs (peptide nucleic acids), phosphorothioates, phosphorodiamidates, morpholinos, and other variants of the phosphate backbone of natural polynucleotides.

DNA may be in form of cDNA, synthetically polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, or derivatives of these groups.

A integrated transposon can express an exogenous nucleotide sequence to inhibit, eliminate, augment, or alter expression of an endogenous nucleotide sequence, or to affect a specific physiological characteristic not naturally associated with the cell. The transposon may contain an expression cassette coded to express a whole or partial protein, or RNA. An expression cassette refers to a natural or recombinantly or synthetically produced nucleic acid that is capable of expressing a gene(s). The term recombinant as used herein refers to a nucleic acid molecule that is comprised of segments of polynucleotide joined together by means of molecular biological techniques. The cassette contains the coding region of the gene of interest along with any other sequences that affect expression of the gene. A DNA expression cassette typically includes a promoter (allowing transcription initiation), and a sequence encoding one or more proteins. Optionally, the expression cassette may include, but is not limited to, transcriptional enhancers, non-coding sequences, splicing signals, transcription termination signals, and polyadenylation signals. The cassette may also code for an siRNA, antisense RNA or DNA, or a ribozyme. A siRNA is a nucleic acid that is a short, 15-50 base pairs and preferably 21-25 base pairs, double stranded ribonucleic acid. The siRNA consists of two annealed strands of RNA or a single strand of RNA that is present in a stem-loop. The siRNA contains sequence that is identical or nearly identical to a portion of a gene. RNA may be polymerized in vitro, recombinant RNA, contain chimeric sequences, or derivatives of these groups. An anti-sense polynucleotide is a polynucleotide that interferes with the function of DNA and/or RNA. Interference may result in suppression of expression. The polynucleotide can also be a sequence whose presence or expression in a cell alters the expression or function of cellular genes or RNA.

A functional RNA comprises any RNA that is not translated into protein but whose presence in the cell alters the endogenous properties of the cell. RNA function inhibitors can cause the degradation of or inhibit the function or translation of a specific cellular RNA, usually a mRNA, in a sequence -specific manner. Inhibition of an RNA can thus effectively inhibit expression of a gene from which the RNA is transcribed. Functional RNAs may be selected from the group comprising: siRNA, interfering RNA or RNAi, dsRNA, RNA Polymerase III transcribed DNAs, ribozymes, and antisense nucleic acid. SiRNA comprises a double stranded structure typically containing 15-50 base pairs and preferably 21-25 base pairs and having a nucleotide sequence identical or nearly identical to an expressed target gene or RNA within the cell. Antisense RNA comprise sequence that is complimentary to an mRNA. RNA polymerase III transcribed DNAs contain promoters, such as the U6 promoter. These DNAs can be transcribed to produce small hairpin RNAs in the cell that can function as siRNA or linear RNAs that can function as antisense RNA.

The transposon may contain an expression cassette encoded to express a whole or partial protein. The protein can be missing or defective in an organism as a result of genetic, inherited or acquired defect in its genome. For example, a polynucleotide may be coded to express the protein dystrophin that is missing or defective in Duchenne muscular dystrophy. Subsequently, dystrophin is produced by the formerly deficient cells. Other examples of imperfect protein production that can be treated with gene therapy include the addition of the protein clotting factors that are missing in the hemophilias and enzymes that are defective in inborn errors of metabolism such as phenylalanine hydroxylase. A delivered polynucleotide can also be therapeutic in acquired disorders such as neurodegenerative disorders, cancer, heart disease, and infections. A therapeutic effect of the protein in attenuating or preventing the disease state can be accomplished by the protein either staying within the cell, remaining attached to the cell in the membrane or being secreted and dissociating from the cell where it can enter the general circulation and blood. Secreted proteins that can be therapeutic include hormones, cytokines, growth factors, clotting factors, anti-protease proteins (e.g. alpha-antitrypsin) and other proteins that are present in the blood. Proteins on the membrane can have a therapeutic effect by providing a receptor for the cell to take up a protein or lipoprotein. For example, the low density lipoprotein (LDL) receptor could be expressed in hepatocytes and lower blood cholesterol levels and thereby prevent atherosclerotic lesions that can cause strokes or myocardial infarction. Therapeutic proteins that stay within the cell can be enzymes that clear a circulating toxic metabolite as in phenylketonuria. They can also cause a cancer cell to be less proliferative or cancerous (e.g. less metastatic). A protein within a cell could also interfere with the replication of a virus.

The term gene generally refers to a nucleic acid sequence that comprises coding sequences necessary for the production of a therapeutic nucleic acid (e.g., siRNA or ribozyme) or a therapeutic polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term encompasses the coding region of a gene. The term may also include sequences located adjacent to the coding region on both the 5′ and 3′ ends for a distance of about 1 kb or more. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences influence the level or rate of transcription and translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, and immunogenicity. One example of covalent modification of nucleic acid involves the action of LabelIT reagents (Mirus Corporation, Madison, Wis.).

As used herein, the term gene expression refers to the process of converting genetic information encoded in a gene into RNA (e.g., mRNA, rRNA, tRNA, snRNA, siRNA, antisense RNA, or ribozyme RNA) through transcription (e.g., via the enzymatic action of an RNA polymerase); and for protein encoding genes, into protein through translation of mRNA.

The term expression cassette refers to a natural or recombinantly produced nucleic acid molecule that is capable of expressing a gene. An expression cassette typically includes a promoter (allowing transcription initiation by either RNA polymerase II or RNA polymerase III), and a sequence encoding one or more proteins or RNAs. Optionally, the expression cassette may include transcriptional enhancers, non-coding sequences, splicing signals, transcription termination signals, and polyadenylation signals, translation termination signals, internal ribosome entry sites (IRES), and non-coding sequences. A nucleic acid can be used to modify the genomic or extrachromosomal DNA sequences. This can be achieved by delivering a nucleic acid that is expressed. Alternatively, the nucleic acid can effect a change in the DNA or RNA sequence of the target cell.

The transposon may contain sequences that do not serve a specific function in the target cell but are used in the generation of the nucleic acid. Such sequences include, but are not limited to, sequences required for replication or selection of the nucleic acid in a host organism.

The terms naked nucleic acid and naked polynucleotide indicate that the nucleic acid or polynucleotide is not associated with a transfection reagent or other delivery vehicle that is required for the nucleic acid or polynucleotide to be delivered to the cell.

A transfection reagent is a compound or compounds that bind(s) to or complex(es) with oligonucleotides and polynucleotides, and mediates their entry into cells. Examples of transfection reagents include, but are not limited to, cationic lipids and liposomes, polyamines, calcium phosphate precipitates, histone proteins, polyethylenimine, and polylysine complexes. It has been shown that cationic proteins like histones and protamines, or synthetic cationic polymers like polylysine, polyarginine, polyomithine, DEAE dextran, polybrene, and polyethylenimine may be effective intracellular delivery agents, while small polycations like spermine are ineffective. Typically, the transfection reagent has a net positive charge that binds to the oligonucleotide's or polynucleotide's negative charge. The transfection reagent mediates binding of oligonucleotides and polynucleotides to cells via its positive charge (that binds to the cell membrane's negative charge) or via cell targeting signals that bind to receptors on or in the cell. For example, cationic liposomes or polylysine complexes have net positive charges that enable them to bind to DNA or RNA. Polyethylenimine, which facilitates gene transfer without additional treatments, probably disrupts endosomal function itself.

The process of delivering a nucleic acid to a cell has been commonly termed transfection or the process of transfecting and has also been termed transformation. The term transfecting as used herein refers to the introduction of foreign nucleic acid or other biologically active compound into cells. The biologically active compound could be used to produce a change in a cell that can be therapeutic. The delivery of nucleic acid for therapeutic and research purposes is commonly called gene therapy. The delivery of nucleic acid can lead to modification of the genetic material present in the target cell. The term stable transfection or stably transfected generally refers to the introduction and integration of exogenous nucleic acid into the genome of the transfected cell. The term stable transfectant refers to a cell which has stably integrated foreign nucleic acid into the genomic DNA. The term transient transfection or transiently transfected refers to the introduction of foreign nucleic acid into a cell where the foreign nucleic acid does not integrate into the gnome of the transfected cell.

### EXAMPLES

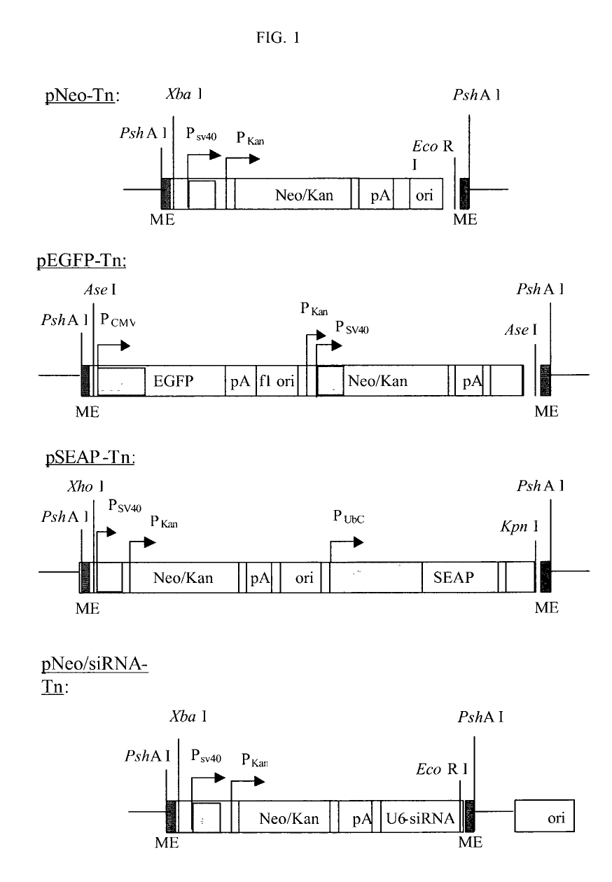

1) Formation of transgene constructs containing transposable elements: DNA sequences to be integrated into the mammalian chromosome were constructed in plasmid vectors. The DNA sequence located between the Tn5 elements plus the Tn5 elements themselves is the transposon. Tn5 elements can be the outer elements (ends), inner elements (ends), or mosaics of outer and inner elements. The mosaic elements (ME) are 19 bp inverted repeats that flank the DNA to be transposed (SEQUENCE ID 1). Precisely at the end of the ME are PshA I restriction sites that allow the transposon DNA to be separated from the plasmid. Internal to the ME are suitable restriction sites that allow removal of the elements. Some examples of transposon constructs are shown in FIG. 1. All of the examples in FIG. 1 include the neomycin/kanamycin resistance gene with SV40 promoter and polyadenylation signal for expression in eukaryotic cells and the prokaryotic Tn5 promoter to drive expression in bacterial cells. Eukaryotic expression allows for selection of mammalian cells that have the integrated transposon. Prokaryotic expression allows for growth of the plasmid in bacterial cells. The origin of replication for bacterial amplification of the plasmids can be included in the transposon as in pNeo-Tn, pSEAP-Tn and pEGFP-Tn. Inclusion of the origin allows for plasmid rescue to determine the integration site in the mammalian genome. The origin can also be in the vector but outside of the transposon sequence, as in pNeo-siRNA-Tn. The vector sequence outside of the Tn5 ME is called the plasmid backbone. The backbone in pNeo-Tn, pSEAP-Tn and pEGFP-Tn is ˜200 bp. The backbone of pNeo-siRNA-Tn is ˜700 bp.

A) Transposon with mosaic element sequence elements:

CTGTCTCTTATACACATCT-(N)ₓ-AGATGTGTATAAGAGACAG The mosaic sequences are underlined (SEQ ID 3 and SEQ ID 4). SEQ ID 4 is the inverted repeat of SEQ ID 3. (N)ₓ represents a sequence that is inserted between the flanking mosaic sequences.

B) Transposon Plasmid pNeo-Tn (FIG. 1)—Plasmid pNeo-Tn for transposition studies was constructed from plasmid pcDNA3 by insertion of the prokaryotic Tn5 promoter between the SV40 promoter and the neomycin/kanamycin resistance (Neo^{R}/Kan^{R}) gene, insertion of two Tn5 transposition mosaic elements (ME), and removal of the ampicillin gene, the CMV promoter, the bovine growth hormone poly A signal and the fl ori. The Tn5 elements flank the sequences to be transposed and are inverted repeats. pNeo-Tn allows for selection with kanamycin in prokaryotic cells and with G418 in eukaryotic cells. pNeo-Tn as shown in FIG.1 is also called pMIR117 and is 2,914 bp. pNeo-Tn without the Xba I site internal to one of the Tn5 elements is called pMIR3 and is 2,913 bp.

C) Transposon Plasmid pSEAP-Tn (FIG. 1)—First, restriction sites were inserted beside one of the mosaic element of pNeo-Tn, using site-directed PCR mutagenesis. The resultant plasmid was pMIR117. The Eco R I/BstB I fragment of pMIR117, containing both mosaic elements and eukaryotic and prokaryotic promoters upstream of the neomycin/kanamicin^{R} gene, was ligated to an EcoR I/BstB I fragment of pMIR7 containing the HSV thymidine kinase polyadenylation signal and an origin of replication. The resultant plasmid was pMIR123. An Sse8387 I restriction site was then inserted into pMIR123, resulting in pMIR124. An EcoR I/Sse8387 I fragment containing the human ubiquitin C promoter, 5′ untranslated region and intron, SEAP cDNA, and SV40 polyadenylation signal from pMIR90 was then inserted into pMIR124, resulting in pMIR126. An internal PshA I site was removed by site-directed mutagenesis to result in pSEAP-Tn, also called pMIR136. pSEAP-Tn expresses human secreted alkaline phosphatase and it is 5,886 bp.

D) Transposon Plasmid pEGFP-Tn (FIG. 1)—Plasmid pEGFP-Tn has the cytomegalovirus (CMV) promoter driving expression of enhanced green fluorescent protein (EGFP); a bacterial origin of replication (ori); and the neomycin/kanamycin resistance gene with an SV40 promoter for expression in mammalian cells and a prokaryotic promoter for expression in bacterial cells. These sequences are flanked by the 19 bp mosaic Tn5 transposition elements. pEGFP-Tn was formed by inserting into the Ase I site of pEGFP-C1 (CLONTECH) a PCR fragment of plasmid pNeo-Tn5 containing the two Tn5 elements separated by a 232 bp backbone and flanked by restriction enzyme Ase I linkers. This plasmid is 5,077 bp.

E) Transposon Plasmid pNeo-siRNA-Tn (FIG. 1)—Plasmid pNeo-siRNA-Tn has the human U6 snRNA promoter for driving expression of siRNA. This plasmid is also called pMIR246. Restriction sites just downstream of the U6 promoter allow for a variety of siRNA sequences to be inserted into pMIR246. The siRNA sequence is determined by the desired target gene.

F) Transposon Plasmid pNeo-U1-Tn—The U6 siRNA expression cassette from pNeo-siRNA-Tn is replaced by two tandem U1 snRNA genes that each target the same mRNA to inhibit its expression.

2) Formation of preformed integrator complexes: Plasmids pNeo-Tn (Example 1B), pSEAP-Tn (Example 1C), and pEGFP-Tn (Example 1D) were purified with the QIAGEN Endo-free maxi-prep kit. Transposon DNA was released from the plasmid backbone by linearization with PshA I and the enzyme was removed by a QIAGEN QIAquick spin column. Concentrations of DNA and transposase were varied to maximize formation of complexes containing one DNA molecule and two Tn5 transposase molecules while minimizing aggregation. Transposase-DNA complexes are preformed by incubating hyperactive mutant Tn5 transposase (53 kDa) in 1× Reaction Buffer (50 mM NaCl, 20 mM HEPES, pH 7.5) with PshA I linearized transposon DNA in a total volume of 20 μl as described in [Goryshin et al. 2000]. The transposase was used at a molar excess of 5- to 10-fold in a reaction volume sufficiently dilute to minimize formation of aggregates. Synaptic complexes were formed by incubation for 2 hours at 37° C. For delivery to mammalian cells, the complexes were concentrated and rinsed twice in a Microcon-100 microfiltration device, thereby replacing the reaction buffer with a physiological buffer and washing out most of the free transposase molecules. Samples of linear or supercoiled transposon DNA alone were prepared at the same DNA concentration. For the DNA sample without mosaic elements, but including transposase, the transposon plasmid was digested with restriction enzymes just internal to the Tn5 ME's. The large fragment was gel purified and added to Tn5 transposase in a mock reaction for complex formation. This mixture was rinsed and concentrated in Microcon-30's, however, because the uncomplexed transposase would filter through a Microcon-100.

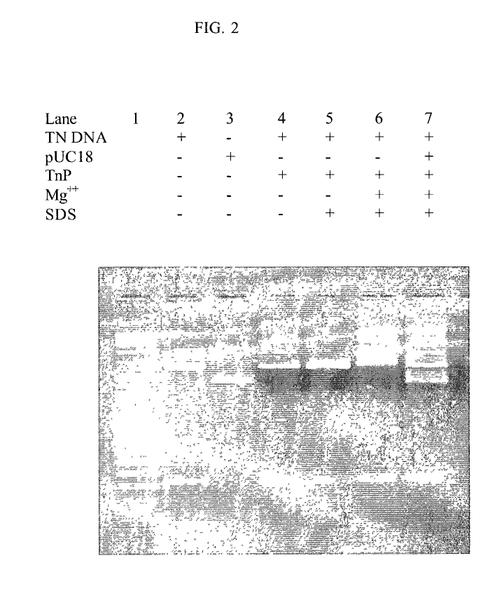

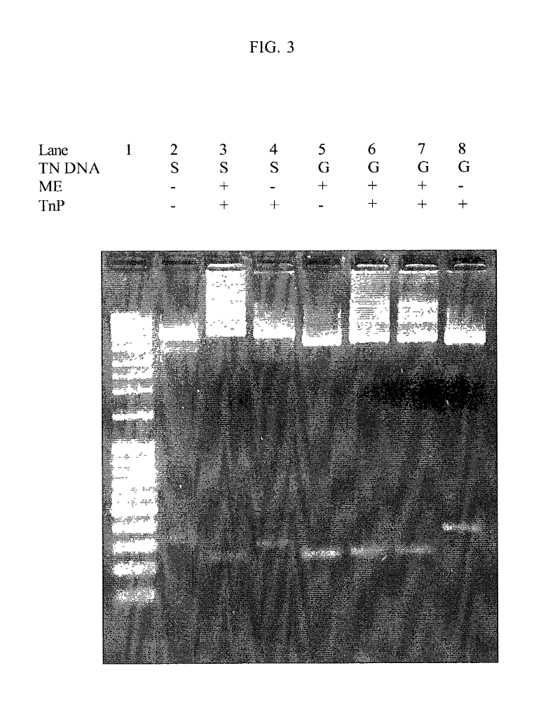

Complexes were then analyzed by agarose gel electrophoresis and ethidium bromide staining to determine how much of the DNA was complexed with transposase. Transposition complex formation with plasmid pNeo-Tn is shown in FIG. 2, lane 4. Transposition complex formation with pSEAP-Tn is shown in FIG. 3, lane 3. Transposition complex formation with pEGFP-Tn is shown in FIG. 3, lanes 6 and 7.

3) Stability of integrator complexes in mammalian transfection reagents: We utilized gel shift assays to evaluate the stability of hyperactive Tn5 transposase binding to linear or supercoiled transposon DNA. Integrator complexes of supercoiled or PshA I-linearized plasmid pSEAP-Tn (FIG. 1) and the hyperactive Tn5 transposase were formed as described above and then incubated with TransIT-LT1, TransIT-HeLaMONSTER®, TransIT-Insecta, PLL, PEI, or Lipofectin transfection reagent in either PBS, Opti-MEM (Invitrogen) or complete media for 1-4 hours.

A. TransIT®-HeLaMONSTER™ (Mirus Corporation): 0.6 μl HeLa reagent was added to the complexes. This mixture was incubated for 10 minutes at ambient temperature.

Then 2 μl of a 10-fold dilution of MONSTER reagent was added.

B. TransIT®-LTI (Mirus Corporation): 0.6 μl reagent was added to the complexes and this mixture was incubated for 10 minutes at ambient temperature.

C. TransIT®-Insecta (Mirus Corporation): 0.8 μl reagent was added to the complexes and the mixture was incubated for 5 minutes at ambient temperature.

D. Lipofectin® (Life Technologies): 0.25 μl reagent was added to the complexes and the mixture was incubated for 15 minutes at ambient temperature.

E. Poly-L-lysine: 0.4 μl of 1 mg/ml reagent was added to the complexes.

F. Linear polyethylenimine (PEI): 0.2 μl of 10 mg/ml reagent was added to the complexes.

An aliquot of each reaction was then transferred to transposase reaction buffer containing pUC18 as a target to determine transposase activity. 150 ng target DNA (pUC18) and 5 μl 5× Activity Assay Buffer were added. The reaction was incubated at 37° C. for 30 minutes. To dissociate transposase from the DNA, 2 μl 5% SDS was added to the reaction and then it was heated at 68° C. for 5 minutes prior to running on an agarose gel for analysis of integration products. Components of both TransIT-HeLaMONSTER and TransIT-LT1 were not fully displaced by SDS treatment. To separate the nucleic acid components from proteins and other polycations, transposition reactions were phenol/chloroform extracted and ethanol precipitated. Reactions that included TransIT-HeLaMONSTER™ , TransIT-LT1 or PLL were treated with 4 μl 0.025% trypsin prior to phenol extraction.

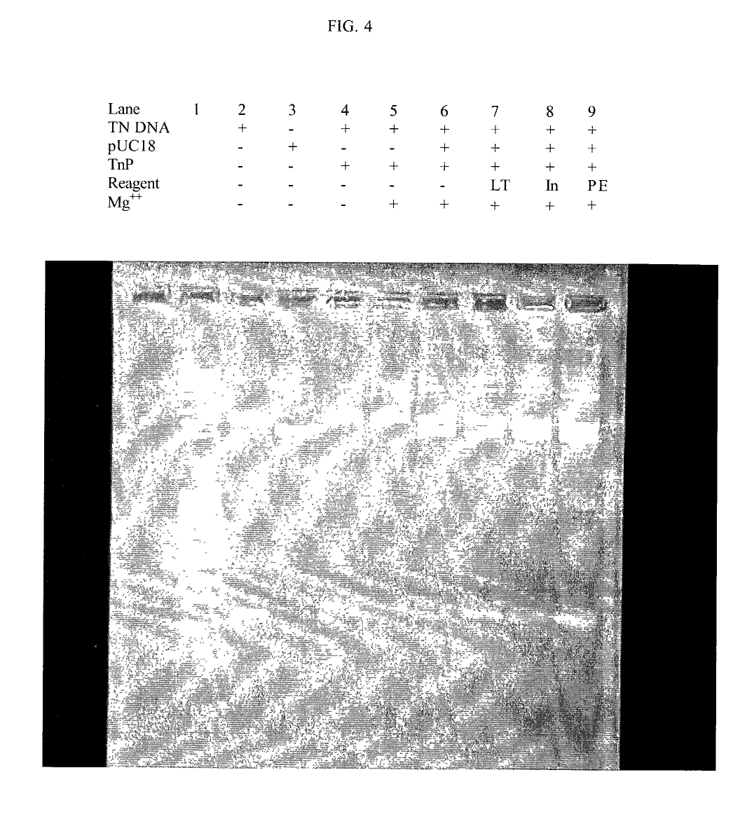

Transposition complexes were treated with TransIT LT1 (LT, FIG. 4, lane 7), Insecta (In, FIG.4, lane 8), PEI (PE, FIG. 4, lane 9), or left untreated (FIG. 4, lane 6). After the integration reaction, the nucleic acids from each reaction were phenol/chloroform extracted and ethanol precipitated as described above. Recovered DNA samples from these reactions are shown in FIG. 4. Integration products from the Tn5 transposition are present in reactions that included no transfection reagent (FIG.4, lane 6) as well as in reactions that occurred in the presence of LT1, Insecta or PEI These results show that the transposase is active in the presence of the transfection reagents.

4) Transfection of preformed transposition complexes into mammalian cells results in an increase in integration events: To demonstrate efficacy of the Tn5 transposase system in effecting integration of a transgene in mammalian cells, NIH3T3 cells were transfected with hyperactive Tn5 transposase complexed with transposon DNA encoding the neomycin resistance gene (pNeo-Tn, pMIR3). As controls, uncut plasmid alone, pCI-Luc⁺ (a luciferase vector not encoding neo^{R}), and linearized transposon DNA were also tested. Cells were plated in 35 mm dishes at 30% confluence in DMEM+10% fetal bovine serum. Linear DNA samples were generated by PshA I restriction enzyme digestion of pNeo-Tn5 and purified with QIAGEN QIAquick spin columns. The small donor backbone fragment was not removed. Transposase-DNA complexes were formed by incubation of linear DNA molecules with a 10-fold excess of transposase. After a two hour incubation, the reaction was concentrated with a Microcon-100 and the complexes were rinsed twice with 20 mM Hepes buffer.

The transfection reagent, TransIT-LT1 (Mirus; Madison, Wis.), was mixed with Opti-MEM and incubated for 5 min. Transposition complexes or DNA alone were then added and the mixtures was incubated for an additional 5 min. Cells were transfected with:

a) 2 μg uncut plasmid (pCI-Luc or transposon plasmid),

b) 2, 5 or 10 μg linear transposon DNA,

c) transposase+1, 2.5 or 5 μg linear transposon DNA,

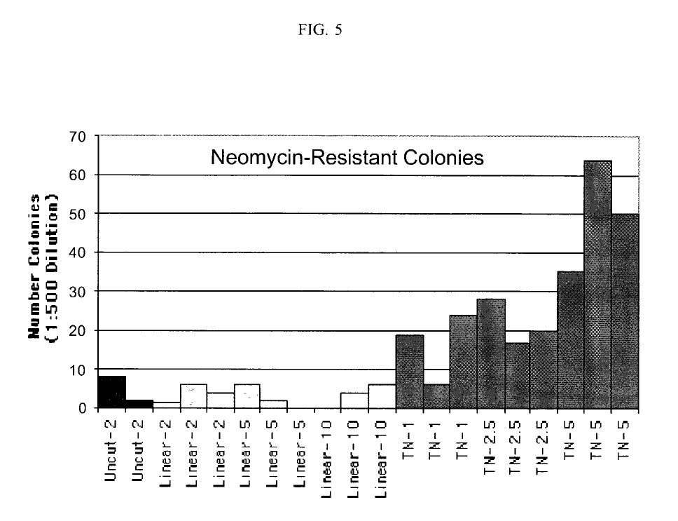

For each condition, 2 μg transfection reagent and 75 μl Opti-MEM were used for each μg DNA. After two days cells were harvested and diluted 1:500 into complete media containing 0.45 mg/ml G418. Colonies were counted after 9 days. These results indicate that Tn5 transposase mediated integration into mammalian cells (FIG. 5).

In this experiment using the transfection reagent TransIT®-LT1 for delivery of integrator complexes or linear DNA alone into NIH3T3 cells, an average of 13 times more neomycin-resistant (neo^{R}) colonies resulted from transfection of the Tn5 transposase-DNA complexes. Transfection of 2, 5 or 10 micrograms linear DNA resulted in approximately equal numbers of neo^{R} colonies, whereas transfection of 1, 2.5 or 5 micrograms of linear DNA with Tn5 transposase complexes resulted in increasing numbers of colonies with increasing amounts of DNA.

5) Integration of human Factor IX gene into the genome of NIH3T3 cells after delivery of hF9-Tn-transposition complexes into NIH-3T3 cells: Trypsinized 3T3 cells were resuspended in PBS at a concentration of 5×10⁶ cells/ml, and 0.7 ml aliquots are transfected with preformed integrator complexes containing a Neo/hF9 transposon as described in example 4. Cells with integrated transposon are selected by adding G418 to the media two days after transfection. The following combinations are used:

(A) PshA I linearized plasmid pNeo/hF9-Tn, which has Tn5 mosaic elements at both ends of the linear DNA encoding the neo^{R} gene (the transposon DNA)

(B) linear transposon DNA complexed to the Tn5 transposase

(C) pNeo/hF9-Tn uncut plasmid, or

(D) Tn5 transposase plus linearized pNeo/hF9-Tn cut with EcoR I and Xba I to remove the Tn5 recognition elements.

Complexes are prepared as described above.

6) Integration of transposon DNA into liver hepatocytes in vivo after injection of transposition complexes into tail vein of mouse: Transposon plasmid pMIR242-Tn has the Tn5 mosaic elements flanking a human factor IX expression cassette. This expression cassette consists of the mouse alpha-fetoprotein enhancer II, mouse albumin promoter with G-52A point mutation, human factor IX cDNA with a truncated intron 1, and the human albumin 3′ untranslated region (UTR) with a truncated intron. The bacterial origin of replication is external to the Tn5 elements as in pNeo-siRNA-Tn (FIG. 1). Transposition complexes are formed with linear transposon DNA, transposon DNA without elements, and on supercoiled plasmid transposon DNA as described in example 4 above. Complexes are delivered in vivo into hepatocytes as described in [Zhang et al. 1999], and employed as a mechanism to treat hemophilia in a mouse model.

7) Integration of SEAP-Tn into the genome after co-transfection of pSEAP-Tn and Tn5 transposase gene into NIH3T3 cells in vitro:

A. Tn5 transposase in a eukaryotic expression vector—The coding sequence of the hyperactive mutant Tn5 transposase (EK54, MA56, LP372) was inserted into Nco I/Acc I of pCI manmmalian expression vector (Promega, Madison, Wis.). The resulting plasmid is pMIR86 (pCMV-Tn5).

B. Tn5 transposase expressed in eukaryotic cells is active—A plasmid-to-plasmid transposition assay was used to determine that the Tn5 transposase was functional in eukaryotic cells. Plasmid DNA was transfected into NIH3T3 cells with TransIT-LT1. This plasmid DNA was composed of transposon plasmid pMIR3 and transposase-encoding plasmid pMIR86. pMIR3 encodes kanamycin resistance in bacteria and pMIR86 encodes ampicillin resistance. Total DNA was harvested from 3T3 cells 25 hours after transfection. This DNA was then transformed into electrocompetent DH10B E. coli cells. The transformed cells were plated on LB-kanamycin plates and then replica-plated onto LB-ampicillin. Plasmid DNA was prepared from individual colonies on the LB-ampicillin plates. Plasmids that were the expected size of a pMIR3 transposon insertion into pMIR86 were sequenced. The transposon insertion sites in the target plasmid showed the characteristic 9 bp direct repeats that prove integration occurred by the Tn5 transposase mechanism. Sequences of the insertion sites are shown below. Lower case bases are from pMIR86. Upper case bases are from transposon pMIR3. Lowercase underlined sequence is the 9 bp duplication of vector sequence at the insertion site. The Tn5 mosaic elements are shown in underlined uppercase letters. Interior of transposon sequence is not shown and is indicated by an underscore.

| | |
|---|---|
| 1. Clone PP5-2: | |
| cggacaggtatccggtaagcggcagggtcggaacaggagCTGTCTCTTATACACATCTAGGGTGT | (SEQ ID 5) |
| GGAAAG TTTTGGTCATGAGAATTCAGATGTGTATAAGAGACAGga | |
| acaggagagcgcacgagggagcttcca. | |
| 2. Clone PP5-7: | |
| accggataaggcgcagcggtcgggctgaacgggggCTGTCTCTTATACACATCTGAATTCTCAT | (SEQ ID 6) |
| GACCAAAA GACTTTCCACACCCTAGATGTGTATAAGAGACAGgaa | |
| cggggggttcgtgcacacagcccagctt. | |
| 3. Clone PP5-8: | |
| gcggtatttcacaccgcatatggtgcactcCTGTCTCTTATACACATCTAGGGTGTGGAAAGT | (SEQ ID 7) |
| CCCCAGGC TTGGTCATGAGAATTCAGATGTGTATAAGAGACAG | |
| ggtgcactctcagtacaatctgctctgatg. | |

C. Tn5 transposase expressed in eukaryotic cells increases the number of integration events. Plasmid pMIR86 encoding transposase was co-transfected into NIH3T3 cells with transposon plasmid pMIR136 (pSEAP-Tn). The pMIR136 was either linearized with PshA I to form the transposon (SEAP-Tn), or cut with Eco R V/Sse8387 I to remove Tn5 elements from the transposon (SEAP), or left supercoiled (pSEAP-Tn). Control reactions included the same transposon DNA samples but with pCI-Luc (Promega) instead of pMIR86. Results in Table I show that reactions containing transposon with elements intact and with transposase plasmid pMIR86 generated about two times as many cell colonies with stably integrated transgene as the reactions that included control plasmid pCI-Luc instead of pMIR86. No increase in colonies was observed when linear transposon DNA lacking flanking recognition elements was used.

TABLE 1

| | Co-transfected | colonies | Ratio |
|---|---|---|---|
| Transposon DNA | plasmid | (×10³) | pMIR86/pCI-Luc |
| Supercoiled pSEAP-Tn | transposase | 547 | 2.1 |
| | control | 258 | |
| SEAP-Tn | transposase | 273 | 1.6 |
| | control | 173 | |
| SEAP | transposase | 77 | 0.9 |
| | control | 90 | |

8) Integration of hF9-Tn into the genome after co-transfection of pMIR242-Tn and Tn5-NLS transposase gene into NIH3T3 cells in vitro:

A. Tn5 transposase with the human importin alpha IBB nuclear localization signal domain: The IBB domain nuclear localization signal (NLS) was cloned downstream of the coding sequence of Tn5 transposase in pMIR86 to be expressed on the carboxyl terminus of the transposase.

Coding sequence for Tn5 transposase-IBB, NLS is in lower case letters (SEQ ID 8):

ATGATAACTTCTGCTCTTCATCGTGCGGCCGACTGGGCTAAATCTGTGTT
CTCTTCGGCGGCGCTGGGTGATCCTCGCCGTACTGCCCGCTTGGTTAACG
TCGCCGCCCAATTGGCAAAATATTCTGGTAAATCAATAACCATCTCATCA
GAGGGTAGTAAAGCCGCCCAGGAAGGCGCTTACCGATTTATCCGCAATCC
CAACGTTTCTGCCGAGGCGATCAGAAAGGCTGGCGCCATGCAAACAGTCA
AGTTGGCTCAGGAGTTTCCCGAACTGCTGGCCATTGAGGACACCACCTCT
TTGAGTTATCGCCACCAGGTCGCCGAAGAGCTTGGCAAGCTGGGCTCTAT
TCAGGATAAATCCCGCGGATGGTGGGTTCACTCCGTTCTCTTGCTCGAGG
CCACCACATTCCGCACCGTAGGATTACTGCATCAGGAGTGGTGGATGCGC
CCGGATGACCCTGCCGATGCGGATGAAAAGGAGAGTGGCAAATGGCTGGC
AGCGGCCGCAACTAGCCGGTTACGCATGGGCAGCATGATGAGCAACGTGA
TTGCGGTCTGTGACCGCGAAGCCGATATTCATGCTTATCTGCAGGACAAA
CTGGCGCATAACGAGCGCTTCGTGGTGCGCTCCAAGCACCCACGCAAGGA
CGTAGAGTCTGGGTTGTATCTGTACGACCATCTGAAGAACCAACCGGAGT
TGGGTGGCTATCAGATCAGCATTCCGCAAAAGGGCGTGGTGGATAAACGC
GGTAAACGTAAAAATCGACCAGCCCGCAAGGCGAGCTTGAGCCTGCGCAG
TGGGCGCATCACGCTAAAACAGGGGAATATCACGCTCAACGCGGTGCTGG
CCGAGGAGATTAACCCGCCCAAGGGTGAGACCCCGTTGAAATGGTTGTTG
CTGACCAGCGAACCGGTCGAGTCGCTAGCCCAAGCCTTGCGCGTCATCGA
CATTTATACCCATCGCTGGCGGATCGAGGAGTTCCATAAGGCATGGAAAA
CCGGAGCAGGAGCCGAGAGGCAACGCATGGAGGAGCCGGATAATCTGGAG
CGGATGGTCTCGATCCTCTCGTTTGTTGCGGTCAGGCTGTTACAGCTCAG
AGAAAGCTTCACGCCGCCGCAAGCACTCAGGGCGCAAGGGCTGCTAAAGG
AAGCGGAACACGTAGAAAGCCAGTCCGCAGAAACGGTGCTGACCCCGGAT
GAATGTCAGCTACTGGGCTATCTGGACAAGGGAAAACGCAAGCGCAAAGA
GAAAGCAGGTAGCTTGCAGTGGGCTTACATGGCGATAGCTAGACTGGGCG
GTTTTATGGACAGCAAGCGAACCGGAATTGCCAGCTGGGGCGCCCTCTGG
GAAGGTTGGGAAGCCCTGCAAAGTAAACTGGATGGCTTTCTTGCCGCCAA
GGATCTGATGGCGCAGGGGATCAAGATCgtcgactccaccaacgagaatg
ctaatacaccagctgcccgtcttcacagattcaagaacaagggaaaagac
agtacagaaatgaggcgtcgcagaatagaggtcaatgtggagctgaggaa
agctaagaaggatgaccagatgctgaagaggagaaatgtaagctcatttc
ctgattga

B. Tn5 transposase with SV40 long NLS: The SV40 long NLS was cloned dowrstream of the coding sequence of Tn5 transposase in pMIR86 to be expressed on the carboxyl terminus of the transposase.

Coding sequence for Tn5 transposase-SV40, NLS is lower case letters (SEQ ID 9):

ATGATAACTTCTGCTCTTCATCGTGCGGCCGACTGGGCTAAATCTGTGTT
CTCTTCGGCGGCGCTGGGTGATCCTCGCCGTACTGCCCGCTTGGTTAACG
TCGCCGCCCAATTGGCAAAATATTCTGGTAAATCAATAACCATCTCATCA
GAGGGTAGTAAAGCCGCCCAGGAAGGCGCTTACCGATTTATCCGCAATCC
CAACGTTTCTGCCGAGGCGATCAGAAAGGCTGGCGCCATGCAAACAGTCA
AGTTGGCTCAGGAGTTTCCCGAACTGCTGGCCATTGAGGACACCACCTCT
TTGAGTTATCGCCACCAGGTCGCCGAAGAGCTTGGCAAGCTGGGCTCTAT
TCAGGATAAATCCCGCGGATGGTGGGTTCACTCCGTTCTCTTGCTCGAGG
CCACCACATTCCGCACCGTAGGATTACTGCATCAGGAGTGGTGGATGCGC
CCGGATGACCCTGCCGATGCGGATGAAAAGGAGAGTGGCAAATGGCTGGC
AGCGGCCGCAACTAGCCGGTTACGCATGGGCAGCATGATGAGCAACGTGA
TTGCGGTCTGTGACCGCGAAGCCGATATTCATGCTTATCTGCAGGACAAA
CTGGCGCATAACGAGCGCTTCGTGGTGCGCTCCAAGCACCCACGCAAGGA
CGTAGAGTCTGGGTTGTATCTGTACGACCATCTGAAGAACCAACCGGAGT
TGGGTGGCTATCAGATCAGCATTCCGCAAAAGGGCGTGGTGGATAAACGC
GGTAAACGTAAAAATCGACCAGCCCGCAAGGCGAGCTTGAGCCTGCGCAG
TGGGCGCATCACGCTAAAACAGGGGAATATCACGCTCAACGCGGTGCTGG
CCGAGGAGATTAACCCGCCCAAGGGTGAGACCCCGTTGAAATGGTTGTTG
CTGACCAGCGAACCGGTCGAGTCGCTAGCCCAAGCCTTGCGCGTCATCGA
CATTTATACCCATCGCTGGCGGATCGAGGAGTTCCATAAGGCATGGAAAA
CCGGAGCAGGAGCCGAGAGGCAACGCATGGAGGAGCCGGATAATCTGGAG
CGGATGGTCTCGATCCTCTCGTTTGTTGCGGTCAGGCTGTTACAGCTCAG
AGAAAGCTTCACGCCGCCGCAAGCACTCAGGGCGCAAGGGCTGCTAAAGG
AAGCGGAACACGTAGAAAGCCAGTCCGCAGAAACGGTGCTGACCCCGGAT
GAATGTCAGCTACTGGGCTATCTGGACAAGGGAAAACGCAAGCGCAAAGA
GAAAGCAGGTAGCTTGCAGTGGGCTTACATGGCGATAGCTAGACTGGGCG
GTTTTATGGACAGCAAGCGAACCGGAATTGCCAGCTGGGGCGCCCTCTGG
GAAGGTTGGGAAGCCCTGCAAAGTAAACTGGATGGCTTTCTTGCCGCCAA
GGATCTGATGGCGCAGGGGATCAAGATCgtcgactcagaagaaatgccat
ctagtgatgatgaggctactgctgactctcaacattctactcctccaaaa
aagaagagaaaggtagaagaccccaaggactttccttcagaattgctaag
ttga

### REFERENCES

1. Blomer U, Naldini L, Kafri T, Trono D, Verma I M, Gage F H. Highly efficient and sustained gene transfer in adult neurons with a lentivirus vector. J Virol. 1997 Sep;71(9):6641-6649.

2. Devine S E, Boeke J D. Efficient integration of artificial transposons into plasmid targets in vitro: a useful tool for DNA mapping, sequencing and genetic analysis. Nucleic Acids Res. 1994 Sep 11;22(18):3765-3772.

3. Goryshin I Y, Jendrisak J, Hoffman L M, Meis R, Reznikoff W S. Insertional transposon mutagenesis by electroporation of released Tn5 transposition complexes. Nat Biotechnol. 2000 Jan; 18(1):97-100.

4. Goryshin I Y, Reznikoff W S. Tn5 in vitro transposition. J Biol Chem. 1998 Mar 27;273(13):7367-7374.

5. Gwinn M L, Stellwagen A E, Craig N L, Tomb J F, Smith H O. In vitro Tn7 mutagenesis of Haemophilus influenzae Rd and characterization of the role of atpA in transformation. J Bacteriol. 1997 Dec; 179(23):7315-7320.

6. Haapa S, Taira S, Heikkinen E, Savilahti H. An efficient and accurate integration of mini-Mu transposons in vitro: a general methodology for functional genetic analysis and molecular biology applications. Nucleic Acids Res. 1999 Jul 1;27(13):2777-2784.

7. Ivics Z, Izsvak Z, Minter A, Hackett P B. Identification of functional domains and evolution of Tc1-like transposable elements. Proc Natl Acad Sci U S A. 1996 May 14;93(10):5008-5013.

8. Ivics Z, Hackett P B, Plasterk R H, Izsvak Z. Molecular reconstruction of Sleeping Beauty, a Tc1-like transposon from fish, and its transposition in human cells. Cell 1997 Nov 14;91(4):501-510.

9. Kenna M A, Brachmann C B, Devine S E, Boeke J D. Invading the yeast nucleus: a nuclear localization signal at the C terminus of Ty1 integrase is required for transposition in vivo. Mol Cell Biol 1998 Feb;18(2):1115-1124.

10. Miyagishi M, Taira K. U6 promoter-driven siRNAs with four uridine 3′ overhangs efficiently suppress targeted gene expression in mammalian cells. Nat Biotechnol. 2002 May;20(5):497-500.

11. Pinkert C A, Ornitz D M, Brinster RL , Palmiter R D. An albumin enhancer located 10 kb upstream functions along with its promoter to direct efficiert, liver-specific expression in transgenic mice. Genes Dev. 1987 May;1(3):268-276.

12. Reznikoff W S, Bhasin A, Davies D R, Gorysbin I Y, Mahnke L A, Naumann T, Rayment I, Steiniger-White M, Twining S S. Tn5: A molecular window on transposition. Biochem Biophys Res Commun. 1999 Dec 29;266(3):729-734.

13. Sui G, Soohoo C, Affar E B, Gay F, Shi Y, Forrester W C, Shi Y. A DNA vector-based RNAi technology to suppress gene expression in mammalian cells. PNAS 2002 99: 5515-5520.

14. Weinreich M D, Gasch A, Reznikoff W S. Evidence that the cis preference of the Tn5 transposase is caused by nonproductive multimerization. Genes Dev. 1994 Oct 1;8(19):2363-2374.

15. York D, Welch K, Goryshin I Y, Reznikoff W S. Simple and efficient generation in vitro of nested deletions and inversions: Tn5 intramolecular transposition. Nucleic Acids Res. 1998 26(8):1927-1933.

16. Zhang G, Budker V, Wolff J A. High levels of foreign gene expression in hepatocytes after tail vein injections of naked plasmid DNA. Hum Gene Ther. 1999 Jul 1;10(10):1735-1737.

17. Zhou M, Bhasin A, Reznikoff W S. Molecular genetic analysis of transposase-end DNA sequence recognition: cooperativity of three adjacent base-pairs in specific interaction with a mutant Tn5 transposase. J Mol Biol. 1998 Mar 13;276(5):913-925.

18. Zhou M, Reznikoff W S. Tn5 transposase mutants that alter DNA binding specificity. J Mol Biol. 1997 Aug 22;271(3):362-373.

19. U.S. Pat. No. 6,437,109 System for in vitro transposition

20. U.S. Pat. No. 6,406,896 Transposase enzyme and method for use

21. U.S. Pat. No. 6,294,385 Method for making insertional mutations using a Tn5 synaptic complex

22. U.S. Pat. No. 6,159,736 Method for making insertional mutations using a Tn5 synaptic complex

23. U.S. Pat. No. 5,965,443 System for in vitro transposition

24. U.S. Pat. No. 5,948,622 System for in vitro transposition

25. U.S. Pat. No. 5,925,545 System for in vitro transposition

We Claim:

1. A process for integrating nucleic acid into the genome of mammalian cells comprising, forming an integrator complex between the nucleic acid containing a transposon and a transposase specific for the transposon; and, delivering the integrator complex to a mammalian cell.

2. The process of claim 1 wherein the nucleic acid encodes an expressible gene.

3. The process of claim 2 wherein the expressible gene encodes a protein.

4. The process of claim 2 wherein the expressible gene encodes a functional RNA.

5. The process of claim 4 wherein the functional RNA is a siRNA.

6. A composition for integrating nucleic acids into a mammalian genome, comprising, a Tn5 integrator complex consisting of Tn5 transposase and a mammalian nucleic acid sequence.

7. The process of claim 1 wherein the integrator complex is formed prior to delivery.

8. The process of claim 1 wherein the transposase consists of a hyperactive transposase.

9. The process of claim 8 where in the hyperactive transposase is the EK54, MA56, LP372 transposase.

10. The process of claim 1 wherein the transposase contains a nuclear localization signal.

11. A process for integrating a nucleic acid into amammalianchromosome, comprising: forming an integrator complex in solution; associating the complex with a transfection reagent; delivering the complex to a mammalian cell wherein the nucleic acid is integrated into the mammalian chromosome.

12. The process of claim 11 wherein the transposon contains elements selected from the group consisting of: outer elements, inner elements, and mosaic elements.
